(19) United States
(12) Patent Application Publication
(10) Pub. No.: US 2005/0005954 A1
(43) Pub. Date: Jan. 13, 2005

(54) Apparatus and process for washing drying and sterilising industrial plants

(76) Inventor: Ruggero Barani, Modena (IT)

Correspondence Address: YOUNG & THOMPSON, 745 SOUTH 23RD STREET, 2ND FLOOR, ARLINGTON, VA, 22202, US

(21) Appl. No.: 10/492,364

(22) PCT Filed: Oct. 7, 2002
(86) PCT No.: PCT/IT02/00637

(30) Foreign Application Priority Data
Oct. 16, 2001 (EP) 01830649.8
Oct. 16, 2001 (EP) 01830650.6

(51) Int. Cl. B08B 3/04

(52) U.S. Cl. 134/94.1; 134/166 C; 134/169 C; 134/102.3; 134/95.2

(57) ABSTRACT

A water tank is connected through a high-pressure feed line to one or more washing terminals associated to a surface to be treated. An ozoniser removes oxygen from a tank and transforms it into ozone, which is conveyed to the tank where it is mixed with the water. The ozone is then conveyed to a drying line which takes drying air from the atmosphere and sends it to the surfaces to be treated. The ozone mixes with the drying air, forming a mix which strikes the surfaces of the plant, drying and sterilising them. A secondary sterilising line selectively places the tank in closed-circuit communication with some parts which during normal operation are not crossed by the washing liquid. The invention is particularly suited to the pharmaceutical industry, and enables efficient sterilisation of treated surfaces even where they are not directly contacted by the liquid sprayed from the washing terminals.

### TECHNICAL FIELD

The invention relates to the field of apparatus and methods for washing and sterilising industrial plants.

### BACKGROUND ART

Especially, though not exclusively, the invention is usefully applied in plants in the pharmaceutical industry, such as for example granulation, mixing, dissolving,

U.S. Pat. No. 5,329,950 discloses a self-containing cleanig and sanitizing equipment which includes: a first liquid holding tank for a cleaning solution, a second liquid holding tank for a sanitizing solution, a cleaning line running from the first tank and a sanitizing line running from the second tank. A sterilizing gas is introduced in the sanitizing line.

The main aim of the present invention is to provide an especially efficient process for automatic washing, drying and sterilising of the treated surfaces of an industrial plant.

An advantage is that even the parts which do not come into direct contact with the washing liquid spraying the plant during cleaning operations are fully sterilised.

A further aim is to provide a simple and economical apparatus for realising the above-cited process.

A further aim of the invention is simply and practically to sterilise the apparatus using cold fluids.

These aims and advantages and others besides are all attained by the invention as it is characterised in the appended claims.

### DISCLOSURE OF INVENTION

Further characteristics and advantages of the present invention will better emerge from the detailed description that follows of a preferred but non-exclusive embodiment of the invention, illustrated purely by way of a non-limiting example in the accompanying FIGURE of the drawing, in which:

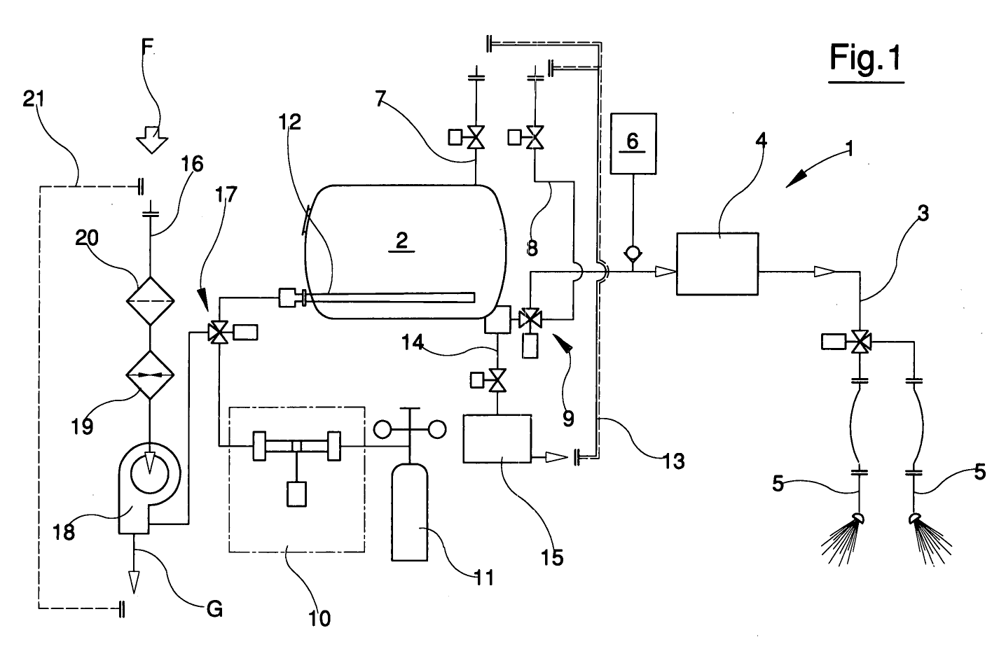

FIG. 1 is a diagram of an apparatus realised according to the invention.

With reference to the FIGURE of the drawing, 1 denotes in its entirety a plant comprising two combined apparatus which cooperate with one another. A first apparatus is for drying and sterilising industrial plants and a second apparatus is for washing industrial plants.

The apparatus comprises a tank 2 containing a washing liquid (for example, water) connected, through a high-pressure feed line 3 comprising a pipe including a pump 4 (for example a high-pressure pump), to one or more washing terminals 5 (for example water wand-type nozzles) which are associated with the surfaces of the plant which are to be treated. In the illustrated embodiment there is also a batcher 6 which feeds the line 3 with measured amounts of a detergent at a point upstream of the pump 4.

A short line 7 is attached to the tank 2, which will join up with an external line supplying the washing fluid, for example the municipal water supply. A further short line 8, also removably connectable to the water supply, joins the high-pressure feed line 3 at a three-way switch valve 9 predisposed (upstream of the high-pressure pump and at a non-pressurised point) selectively to connect the high-pressure feed line 3 with the tank supply 2 or with the municipal water supply through the connecting line 8. Both short connecting lines 7 and 8 are provided with an intercept valve.

The apparatus 1 comprises means for introducing a sterilising gas into the tank 2, which gas mixes with the washing liquid. The means for introducing the sterilising gas comprise a gas feed line which includes an ozoniser 10 (provided with a high-voltage generator). The ozoniser 10 takes oxygen from a tank 11 and transforms it into ozone. Any fixed supply line of oxygen can be used. The oxygen supply line terminates inside the tank 2 with an injector 12 located close to the bottom of the tank 2 which releases the ozone (a sterilising gas) into the washing liquid, thus obtaining a mixture of the two substances.

The apparatus comprises a secondary automatic sterilising line 13 predisposed to connect selectively the tank 2 with parts of the apparatus which during normal functioning are not crossed by the washing liquid, so that these parts too can be sterilised. In the illustrated embodiment these non-contact parts are the two short water lines 7 and 8 which supply the washing liquid (water) to the tank, and the high-pressure feed line 3. During automatic sterilisation the ends of the short lines 7 and 8 are detached from the municipal water supply and joined up to the secondary automatic sterilising line 13. The secondary line 13 exhibits at one end a removable and selective connection with the short lines 7 and 8; while at its other end it exhibits a connector for selective and removable connection with a subsidiary draining line 14 of the tank 2, provided with a draining pump 15, which can on demand be used both for draining liquid remaining in the tank 2 and for automatic sterilisation of parts of the apparatus.

The drying apparatus comprises a drying line 16 predisposed to supply a drying gas to the treated surfaces of the industrial plant (of known type and not illustrated).

The drying line 16 is connected through a three-way switch valve 17 to the feed line which introduces the sterilising gas into the tank; this connection enables the sterilising gas—ozone—to be mixed with the drying gas—air. The drying line 16 is provided with a feed line which removes air from the atmosphere; along the feed line means for moving the air are arranged (for example an aspirating fan), a heater 19 and a filter 20.

The drying gas is sent towards the plant to be dried in the direction of arrow G. The drying line 16 is connected through a conduit and a valve 17 to a gas feed line on which an ozonizer 10 (provided with a voltage generator) is provided. The ozoniser 10 takes oxygen from a tank 11 and transforms it into ozone. Any fixed supply line of oxygen can be used. In the illustrated embodiment the valve is a three-way switch valve 17 to the feed line which introduces the sterilising gas into at least one user (in this embodiment the second washing apparatus, as will be better explained herein below), as well as enabling the sterilising gas—ozone —to be mixed with the drying gas—air before the mixture is sent on to the surfaces to be treated. Predisposing at least one further user for use with the sterilising gas supply is optional, preferable but not necessary.

The drying line 16, like the short lines and 8, is associated to a secondary recycling line 21 which is selectively connectable to the drying line 16, to create a closed automatic sterilising circuit of the drying line 16. The secondary recycling line 21 is basically a by-pass for performing, when so desired, an automatic sterilisation of the drying line 16.

The various above-mentioned parts of the apparatus 1—in particular the tank 2, the high-pressure feed line 3, the pump 4, the washing terminals 5 and the means for introducing sterilising gas, but also the drying system as well as the auto-sterilising systems with ozonised water and ozonised air—are mounted on a mobile washing unit with wheels (not illustrated).

The apparatus enables automatic sterilisation and cleaning of structural components with no need to dismount them: this is realised by means of the on-site production of sterilising gas (ozone) which is mixed with the washing liquid and thus conveyed at low temperature to the inside of the hydraulic circuit of the apparatus. The same solution is applied to the pneumatic drying circuit, with the difference that the sterilising gas is mixed with the drying gas. This solution means that the same control unit can be programmed to command both the washing/drying operations and the automatic sterilising cycle.

Cold automatic sterilising (instead of, for example, sterilising using heated steam) of the washing and drying apparatus means that relatively-cheap parts of the apparatus itself can be used in the operation, as no compatibility with high temperatures is required.

Production and introduction of sterilising gas into the tank 2 and the pneumatic circuit are controlled (using known techniques) so that the saturation level of sterilising gas (in this embodiment ozone) mixed with the washing liquid (in this case water) or in the drying fluid (in this case air) is never exceeded, so no over-production of ozone obtains.

It has been seen that by using a high-pressure washing liquid and a mixed-in sterilising gas (i.e. in the present embodiment ozonised water) an extremely efficient wash and sterilisation is obtained of all of the treated surfaces of the plant. In the case of ozonised water, high-pressure delivery over the surfaces to be treated accelerates the transformation of the ozone contained in the water into active oxygen, which is able to sterilise even those parts of the machine which are not directly sprayed by the jets of pressurised liquids.

The invention has the advantage of concentrating the washing, the drying and sterilising operations in a single compact apparatus which is self-sufficient and mobile in that it can be mounted on wheels. It also enables a final printing of a single non-changeable report to be made of all the washing, drying and sterilisation process.

The invention also consists in the actuation of a drying and sterilising process for industrial plants in which a drying gas is taken from a source, for example the atmosphere, and sent through one or more conduits twoars at least one surface of a plant to be dried and sterilised, which is struck by the drying gas flow. The process introduces a sterilising gas into the conduits, which is mixed with the drying gas.

The drying gas comprises ozone.

The process, which is preferably performed by an apparatus such as the one described, automatically sterilises a part of the apparatus with the drying gas flow in a closed circuit.

1.-21. (cancelled)

22. An apparatus for washing and sterilising industrial plants, comprising at least one tank (2) for a washing liquid connected, through at least one high-pressure feed line (3) equipped with at least one pump (4), to at least one washing terminal (5) which is associated to a surface to be treated, characterized in that it comprises means (10, 11, 12) for introducing a sterilising gas into the tank (2), which sterilising gas mixes with the washing liquid in the tank.

23. The apparatus of claim 22, characterized in that the means for introducing the sterilising gas comprise an ozoniser (10) which removes oxygen from a tank (11) and transforms the oxygen into ozone, which is conveyed to the tank (2).

24. The apparatus of claim 22, characterized in that it comprises at least one secondary line for automatic sterilisation (13) which can selectively place in a closed-circuit communication the tank (2) with parts of the apparatus which during normal functioning are not crossed by the washing liquid, in order that the parts of the apparatus which during normal functioning are not crossed by the washing liquid can be washed.

25. The apparatus of claim 24, characterized in that the parts of the apparatus which are automatically sterilised comprise one or more short lines (7 and 8) for supplying a washing liquid to the tank (2) and the high-pressure feed line (3), each of the short lines (7 and 8) being provided with at least one end which can be attached to an external supply source.

26. The apparatus of claim 22, characterized in that it comprises a drying line (16) for supply a gaseous drying fluid to treated surfaces, which drying line (16) is connected to means for introducing sterilising gases for mixing the sterilising gas with the gaseous drying fluid.

27. The apparatus of claim 26, comprising a secondary line (21) for recycling, selectively connected to the drying line in order to realize a sterilising closed circuit of the drying line (16).

28. The apparatus of claim 22, characterized in that at least the tank (2), the high-pressure feed line (3), the pump (4), the washing terminal (5) and the means (10, 11, 12) for introducing the sterilising gas are mounted on a compact washing unit which is self-sufficient and mobile on wheels.

29. (new) the apparatus of claim 26, characterized in that the means for introducing the sterilising gas comprise an ozoniser (10) which removes oxygen from a tank (11) and transforms the oxygen into ozone, which is conveyed to the drying line (16).

30. The apparatus of claim 22, characterized in that it comprises at least one secondary line for automatic sterilisation (13) which can selectively place in a closed-circuit communication the tank (2) with parts of the apparatus which during normal functioning are not crossed by the washing liquid, in order that the parts of the apparatus which during normal functioning are not crossed by the washing liquid can be washed.

31. The apparatus of claim 22, charactersied in that at least the tank (2), the high-pressure feed line (3), the pump (4), the washing terminal (5) and the means (10, 11, 12) for introducing the sterilising gas are mounted on a compact washing unit which is self-sufficient and mobile on wheels.

32. A process for washing and sterilising industrial plants wherein a washing liquid is sourced from a tank (2), is pumped and sent at high pressure toward at least one washing terminal (5) which directs the washing liquid to a washing and sterilising plant, characterized in that it injects a sterilising gas internally of the tank (2), which sterilising gas mixes with the washing liquid.

33. The process of claim 32, characterized in that the sterilising gas comprises ozone.

34. The process of claim 32, characterized in that, after washing, surfaces of the plant are sprayed with a drying flow of gas containing the sterilising gas.

35. The process of claim 32, characterized in that the process is realized by an apparatus which is automatically sterilised by the closed-circuit drying flow of gas.

36. The process of claim 32, characterized in that the process automatically washes and automatically sterilises, in a closed circuit and uses the washing liquid sourced from the tank (2), at least a part of the apparatus, especially the connections for connecting the tank (2) and the high-pressure feed line (3) with an external source of the washing liquid.
